(19) United States
(12) Patent Application Publication
(10) Pub. No.: US 2017/0185747 A1
(43) Pub. Date: Jun. 29, 2017

(54) INTELLIGENTLY-ANALGESIC INFUSION PUMP MONITORING SYSTEM AND METHOD

(71) Applicant: Weizhong YU, Chengdu, Sichuan (CN)

(72) Inventors: Weizhong YU, Chengdu (CN); Wenkai YU, Chengdu (CN)

(21) Appl. No.: 15/304,864

(22) PCT Filed: Feb. 27, 2015
(86) PCT No.: PCT/CN2015/073385

§ 371 Date: Oct. 18, 2016

(30) Foreign Application Priority Data
Apr. 18, 2014 (CN) 201410156960.7

(51) Int. Cl. G06F 19/00; A61B 5/02; A61B 5/00; A61M 5/168

(52) U.S. Cl. CPC G06F 19/3468; A61M 5/16886; A61M 5/168; A61B 5/02; A61B 5/0002; A61M 2005/1405

(57) ABSTRACT

The present invention discloses a system and method for monitoring an infusion pump capable of intelligently easing pain. Each infusion pump control terminal is connected with a monitoring server through a wireless AP and a local area network respectively; each human body vital sign sensor is connected with the signal input end of a field programmable gate array FPGA through a sensor interface circuit respectively, an infusion control device is connected with the control signal output end of the field programmable gate array FPGA, the field programmable gate array FPGA is in communication with an ARM processor in a bus coding mode, and the ARM processor is in communication connection with the wireless AP through a WIFI communication module. By means of the system and method for monitoring infusion pump capable of intelligently easing pain, a plurality of basic vital sign data of a patient is collected in real time, corresponding infusion schemes are generated through analysis of the data, the infusion pump is controlled to achieve automatic infusion, monitoring and pain-easing infusion are combined together for coordinative work, and infusion control is more scientific and reliable; patient online perception and feedback is supported, self-improvement of a system is facilitated, and more accurate and reliable infusion schemes can be acquired.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

Field of Invention

The present invention relates to a medical appliance, and more particularly to a system and method for monitoring an infusion pump capable of intelligently easing pain.

Description of Related Arts

An electronic infusion pump plays an increasingly important role in relieving the sharp pain of patients after operation, other pain suffered by patients, and improving the life quality of patients; and thus has been widely applied in clinic. Due to individual differences of patients, problems appeared during the application of the infusion pump are relatively apparent. For example, individualized drug application according to the demands of the patients cannot be realized sometimes due to a lack of effective tutelage and real time monitoring, so that risks caused by insufficient or excessive medication cannot be avoided. Insufficient medication will cause incomplete analgesia, while excessive medication will cause respiratory depression, blood pressure drop, all-over pruritus, uroschesis, lethargy and the like, which will even cause threat to life in case of being not found and treated in time. In order to monitor the infusion dose of the patient, a manner of ward rounds manually conducted by doctors is presently employed, which is straggled no matter on timeliness or availability.

Intelligent infusion pumps are developed earlier at abraod. For example, countries like Japan, USA and Germany have developed infusion pumps since late 1980s. Prevalent infusion pumps on the market presently are mostly foreign products, which have various types and better performances. For example, OT-601 infusion pump (the control accuracy is 10%) and SP-500 syringe pump of Japan JMS, GeminiPC-2TX infusion pump of US MED Corporation which can realize four-way control, Multifuse, Perfusor Compact (the control accuracy may reach 2%), Infusomat P and Infusomat fmS of Germany B.BRAUN (B.BRAUN). There are numerous types of infusion pumps, and Isreal also has corresponding products. Infusion pump has been developed in middle 1980s at home, which started relatively late. Although there are some domestic infusion pumps on the market, for example, ZNB series infusion pump products of Shenzhen Kangfute Corporation and Beijing Keilifenggao Science and Technology Development Co., Ltd., the type of domestic infusion pumps is fewer and the performances thereof need to be further improved generally. The infusion pumps will be developed towards an orientation of being more portable, smaller, accurate, safer and reliable to control.

### SUMMARY OF THE PRESENT INVENTION

The object of the present invention aims at overcoming the defects of the prior art, and providing a system and method for monitoring an infusion pump capable of intelligently easing pain, which supports such functions like intellectual infusion control, online monitoring of basic vital sign data of a suffer, online warning of patient online perception and feedback, and the like.

The object of the present invention is fulfilled by employing the following technical solution: a system for monitoring an infusion pump capable of intelligently easing pain includes at least one terminal device unit and a monitoring server, wherein each terminal device unit includes at least one infusion pump control terminal and a wireless AP, wherein each infusion pump control terminal is connected with the monitoring server through the wireless AP and a local area network respectively.

The infusion pump control terminal includes at least one human body vital sign sensor, an infusion control device, a field programmable gate array FPGA, an ARM processor and a WIFI communication module, wherein each human body vital sign sensor is connected with a signal input end of the field programmable gate array FPGA through a sensor interface circuit respectively, the infusion control device is connected with a control signal output end of the field programmable gate array FPGA, the field programmable gate array FPGA is in communication with the ARM processor in a bus coding mode, and the ARM processor is in communication connection with the wireless AP through the WIFI communication module.

The monitoring server includes an infusion scheme input module for providing an infusion scheme input interface for a doctor, and the ARM processor includes an infusion scheme processing module for converting an infusion scheme into an infusion control device control signal.

The human body vital sign sensor includes any one or a combination of a blood pressure sensor, a finger temperature sensor, a pulse sensor, a body temperature sensor and a blood oxygen sensor.

The sensor interface circuit includes an I2C, SPI or RS232 interface circuit.

The infusion pump control terminal further includes an LCD display screen and a warning circuit, wherein the LCD display screen is connected with the ARM processor, and the warning circuit is connected with the field programmable gate array FPGA.

The infusion pump control terminal further includes an input keyboard, wherein the input keyboard is connected with the field programmable gate array FPGA, and the field programmable gate array FPGA includes an online perception feedback information input module for providing an online perception feedback information input interface for a sufferer.

The infusion pump control terminal further includes an infusion flow rate sensor and a liquid medicine level sensor, wherein the infusion flow rate sensor and the liquid medicine level sensor are connected with the signal input end of the field programmable gate array FPGA respectively.

A method for monitoring an infusion pump capable of intelligently easing pain includes following steps of:
(1) Externally connecting an infusion pump control terminal with a human body vital sign sensor, and collecting various human body vital sign data of a sufferer by the human body vital sign sensor;
(2) Converting a signal by a sensor interface circuit read by the sensor into a signal level which can be recognized by a field programmable gate array FPGA, sending the various human body vital sign data of the sufferer collected to the field programmable gate array FPGA, forwarding the data by the field programmable gate array FPGA to an ARM processor, sending by the ARM processor to a wireless AP through a WIFI communication module, and then transmitting the data to a monitoring server through a local area network; and
(3) Drawing up, by a doctor, a best infusion scheme through analysis on the various human body vital sign data of the sufferer at a monitoring server end, and inputting the infusion scheme into the monitoring server through an infusion scheme input module, sending the infusion scheme to the ARM processor of the infusion pump control terminal through the local area network, the wireless AP and the WIFI communication module, generating a control signal by an infusion scheme processing module in the ARM processor on an infusion control device according to the infusion scheme sent by the doctor, and forwarding the control signal to the infusion control device through the field programmable gate array FPGA, so as to realize automatic infusion control of the infusion pump.

The method for monitoring an infusion pump capable of intelligently easing pain further includes a step of inputting an infusion scheme by the sufferer to perform automatic infusion control on the infusion pump.

The method for monitoring an infusion pump capable of intelligently easing pain further includes a step of online perception and feedback, wherein the sufferer inputs online perception and feedback information through an online perception feedback information input module and uploads the online perception and feedback information to the monitoring server.

The method for monitoring an infusion pump capable of intelligently easing pain further includes a step of infusion abnormality warning and a step of infusion completion warning, wherein:

The step of infusion abnormality warning includes an infusion flow rate sensor collecting an infusion flow rate of the infusion pump in real time and judging infusion abnormality when a flow rate value is abnormal, and the field programmable gate array FPGA sending a warning signal to a warning circuit; and

The step of infusion completion warning includes a liquid medicine level sensor collecting a level of residual liquid medicine in the infusion pump in real time and judging infusion completion when the liquid value is lower than a preset threshold, and the field programmable gate array FPGA sending a warning signal to the warning circuit.

The present invention has the advantageous effects that:
(1) a plurality of basic vital sign data of the sufferer such as blood pressure, finger temperature, pulse, body temperature and blood oxygen is collected in real time, corresponding infusion schemes are generated through analysis of the data, the infusion pump is controlled to achieve automatic infusion, monitoring and pain-easing infusion are combined together for coordinative work, and infusion control is more scientific and reliable;
(2) remotely centralized monitoring, management and dispatching of a plurality of infusion pumps may be realized, so that the efficiency and the system reliability are improved;
(3) the online perception and feedback of the sufferer are supported, and the system can be helped for self-improvement, so as to obtain a more accurate and reliable infusion scheme; and
(4) infusion flow rate monitoring and liquid level monitoring are supported, infusion abnormality and infusion completion may be judged through the flow rate and liquid level data, and automatic warning can be performed in case of infusion abnormality or infusion completion, to prompt medical care personnel or sufferer to treat in time, thus improving the infusion safety.

### BRIEF DESCRIPTION OF THE DRAWINGS

FIG. 1 is a network structure topological graph of a monitoring system according to the present invention;

FIG. 2 is a structure block diagram of an infusion pump control terminal according to the present invention; and

FIG. 3 is a flow chart of a monitoring method according to the present invention.

### DETAILED DESCRIPTION OF THE PREFERRED EMBODIMENT

The present invention will be further described hereinafter by reference to the embodiments, but the protection scope of the present invention is not limited to the following descriptions.

As shown in FIG. 1, a system for monitoring an infusion pump capable of intelligently easing pain includes at least one terminal device unit and a monitoring server, wherein each terminal device unit includes at least one infusion pump control terminal and a wireless AP, wherein each infusion pump control terminal is connected with the monitoring server through the wireless AP and a local area network respectively.

One or more wireless APs may be disposed in each ward. The wireless AP has two application manners. The first manner is to set the wireless AP into a bridging mode, convert a wired network to which the wireless AP is accessed into a wireless network, wherein a wireless network gateway and an IP address field are kept unchanged. The quantity of terminals which can be connected with each wireless AP is determined by the quantity of residual IP addresses of the original local area network when using this application manner. The second manner is to set the wireless AP into a routing mode, wherein the wireless APs make up a single wireless local area network, wherein the network is a subnetwork of the wireless AP accessed to the local area network; and the wireless AP is responsible for routing two network addresses. The quantity of the terminals which can be connected with each wireless AP is more than 200 when using this application manner.

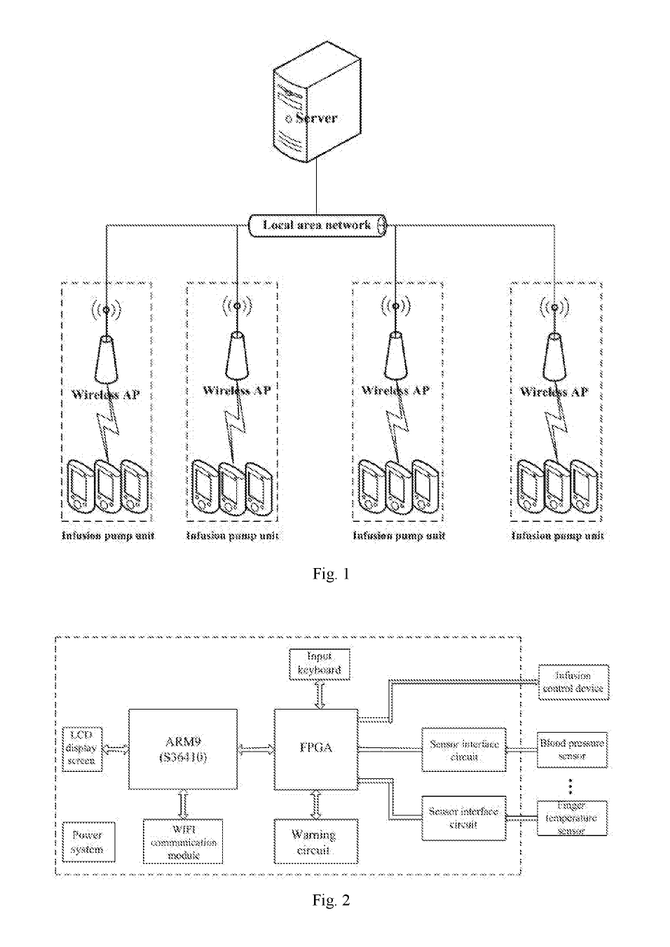

As shown in FIG. 2, the infusion pump control terminal includes at least a human body vital sign sensor, an infusion control device, a field programmable gate array FPGA, an ARM processor and a WIFI communication module, wherein each human body vital sign sensor is connected with a signal input end of the field programmable gate array FPGA through a sensor interface circuit respectively, the infusion control device is connected with a control signal output end of the field programmable gate array FPGA, the field programmable gate array FPGA is in communication with the ARM processor in a bus coding mode, and the ARM processor is in communication connection with the wireless AP through the WIFI communication module. The monitoring server includes an infusion scheme input module for providing an infusion scheme input interface for a doctor, and the ARM processor includes an infusion scheme processing module for converting an infusion scheme into an infusion control device control signal.

An ARM9 536410 processor is employed as the ARM processor, wherein the processor supports embedded operating systems such as Windows CE, Linux and the like, which facilitates software system development. Meanwhile, the processor has low power consumption and abundant interfaces, supports WIFI communication, LAN communication, USB communication and the like, and is very suitable for developing portable mobile instruments and apparatuses. A system clock of the ARM9 536410 can reach as high as 800 MHz, which can satisfy the requirements on real time control and information interaction.

FPGA is employed for controlling between the ARM processor and the sensor, the infusion control device, and the warning circuit, wherein the abundant I/O resources of the FPGA facilitates system upgrading.

An infusion pump driving module of the infusion control device consists of a two-phase four-wire stepping motor and a pump body. Because accurate and reliable metering are required for infusion, the stepping motor is selected as a power plant. The stepping motor is an actuator which converts changes of pulsed excitation into exact rotor position increment motion, i.e., converts a pulse signal into corresponding angular displacement of a motor. Therefore, the running time of the motor can be controlled through the quantity of control pulses, and the running speed of the motor can be controlled through changing a pulse frequency. The running of the motor drives a cam mechanism of the pump body to compress a rubber tube in a circulating manner to realize the infusion function. In order to make the infusion pump work safely and reliably, and start from the angle of simplifying a hardware circuit, the running of the stepping motor is not directly controlled by a Darlington tube driven by a singlechip; instead, a driving module of the stepping motor is indirectly controlled through two control wires, thus realizing start and control respectively. M008335 is selected as a driving chip of the stepping motor herein. A starting signal starts the stepping motor to work, while the running time and stopping time are controlled by a control signal, and the infusion speed is controlled through the difference of the stopping and rotating time of the stepping motor.

The human body vital sign sensor includes any one or a combination of a blood pressure sensor, a finger temperature sensor, a pulse sensor, a body temperature sensor and a blood oxygen sensor.

The sensor interface circuit includes an I2C, SPI or RS232 interface circuit.

The infusion pump control terminal further includes an LCD display screen and a warning circuit, wherein the LCD display screen is connected with the ARM processor, and the warning circuit is connected with the field programmable gate array FPGA.

The infusion pump control terminal further includes an input keyboard, wherein the input keyboard is connected with the field programmable gate array FPGA, and the field programmable gate array FPGA includes an online perception feedback information input module for providing an online perception feedback information input interface for a sufferer. The online perception and feedback information includes: temporarily increased dose (start once in every 24 h, and one drop for each time), analgesia score (VAS), motor score (Bromage), ramsay score (Ramsay), patient sense and the like.

The infusion pump control terminal further includes an infusion flow rate sensor and a liquid medicine level sensor, wherein the infusion flow rate sensor and the liquid medicine level sensor are connected with the signal input end of the field programmable gate array FPGA respectively;

Furthermore, the system for monitoring an infusion pump capable of intelligently easing pain further includes the following functions: (1) input and registration of basic data of the patient as well as operation management based on a wireless network; (2) determining such parameters as infusion frequency (drop number/second), total infusion quantity and total liquid medicine quantity and the like according to the individual conditions of the patient through a basic data analysis system; (3) being capable of changing the originally set parameters in any time and temporarily suspending infusion during running; (4) different users may have a set of self-own operating parameters, and respectively record and dynamically monitor the operating parameters; (5) displaying the vital characters of the sufferer through LCD, and being capable of observing the liquid medicine used and the residual content of the liquid medicine, and reporting to a data center instantly; (6) important operations are both completed by two combination keys to prevent misoperation, so that it is safe and reliable; (7) the user makes requests to a monitoring center instantly, and the monitoring center remotely increases or decreases the medicine dose for the user according to the actual situation, and the sufferer may also press the key at a time for increasing the medicine dose (avaliable for one time in every 24 h); and (8) all the data can be remotely monitored and recorded online; meanwhile, the system has transfinite control and warning functions.

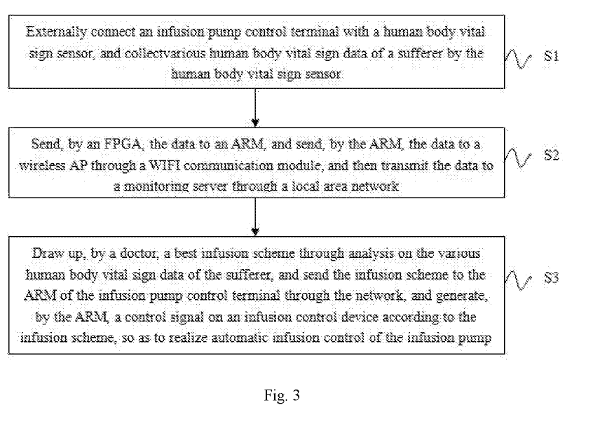

As shown in FIG. 3, the method for monitoring an infusion pump capable of intelligently easing pain includes the following steps of:
(1) Externally connecting an infusion pump control terminal with a human body vital sign sensor, and collecting various human body vital sign data of a sufferer by the human body vital sign sensor;
(2) Converting a signal by a sensor interface circuit read by the sensor into a signal level which can be recognized by a field programmable gate array FPGA, sending the various human body vital sign data of the sufferer collected to the field programmable gate array FPGA, forwarding the data by the field programmable gate array FPGA to an ARM processor, sending by the ARM processor to a wireless AP through a WIFI communication module, and then transmitting the data to a monitoring server through a local area network; and
(3) Drawing up, by a doctor, a best infusion scheme through analysis on the various human body vital sign data of the sufferer at a monitoring server end, and inputting the infusion scheme into the monitoring server through an infusion scheme input module, sending the infusion scheme to the ARM processor of the infusion pump control terminal through the local area network, the wireless AP and the WIFI communication module, generating a control signal by an infusion scheme processing module in the ARM processor on an infusion control device according to the infusion scheme sent by the doctor, and forwarding the control signal to the infusion control device through the field programmable gate array FPGA, so as to realize automatic infusion control of the infusion pump.

The method for monitoring an infusion pump capable of intelligently easing pain further includes a step of inputting an infusion scheme by the sufferer to perform automatic infusion control on the infusion pump.

The method for monitoring an infusion pump capable of intelligently easing pain further includes a step of online perception and feedback, wherein the sufferer inputs online perception and feedback information through an online perception feedback information input module and uploads the online perception and feedback information to the monitoring server.

The method for monitoring an infusion pump capable of intelligently easing pain further includes a step of infusion abnormality warning and a step of infusion completion warning, wherein:

The step of infusion abnormality warning includes an infusion flow rate sensor collecting an infusion flow rate of the infusion pump in real time and judging infusion abnormality when a flow rate value is abnormal, and the field programmable gate array FPGA sending a warning signal to a warning circuit; and

The step of infusion completion warning includes a liquid medicine level sensor collecting the level of residual liquid medicine in the infusion pump in real time and judging infusion completion when the liquid value is lower than a preset threshold, and the field programmable gate array FPGA sending a warning signal to the warning circuit.

The above is merely preferred embodiments of the invention. It should be understood that the invention is not limited to the forms disclosed herein, and shall not be deemed as an exclusion to other embodiments, but can be applied to various other combinations, amendments and circumstances, and can be modified through the foreoging teaching or technologies or knowledge of related arts within the scope of the concept herein. While modifications and changes made by those skilled in the art without departing from the spirit and scope of the invention shall all fall within the protection scope of the claimes of the invention appended.

What is claimed is:

1. A system for monitoring an infusion pump capable of intelligently easing pain, comprising:
at least one terminal device unit and a monitoring server, wherein said each terminal device unit comprises at least one infusion pump control terminal and a wireless AP, wherein said each infusion pump control terminal is connected with said monitoring server through a wireless AP and a local area network respectively;
wherein said infusion pump control terminal comprises at least a human body vital sign sensor, an infusion control device, a field programmable gate array FPGA, an ARM processor and a WIFI communication module, wherein said each human body vital sign sensor is connected with a signal input end of said field programmable gate array FPGA through a sensor interface circuit respectively, wherein said infusion control device is connected with a control signal output end of said field programmable gate array FPGA, wherein said field programmable gate array FPGA is in communication with said ARM processor in a bus coding mode, and said ARM processor is in communication connection with said wireless AP through said WIFI communication module; and
wherein said monitoring server comprises an infusion scheme input module for providing an infusion scheme input interface for a doctor, and said ARM processor comprises an infusion scheme processing module for converting an infusion scheme into an infusion control device control signal.

2. The system, as recited in claim 1, wherein said human body vital sign sensor comprises any one or a combination of a blood pressure sensor, a finger temperature sensor, a pulse sensor, a body temperature sensor and a blood oxygen sensor.

3. The system, as recited in claim 1, wherein said sensor interface circuit comprises an I2C, SPI or RS232 interface circuit.

4. The system, as recited in claim 1, wherein said infusion pump control terminal further comprises an LCD display screen and a warning circuit, wherein said LCD display screen is connected with said ARM processor, wherein said warning circuit is connected with said field programmable gate array FPGA.

5. The system, as recited in claim 1, wherein said infusion pump control terminal further comprises an input keyboard, wherein said input keyboard is connected with said field programmable gate array FPGA, and said field programmable gate array FPGA comprises an online perception feedback information input module for providing an online perception feedback information input interface for a sufferer.

6. The system, as recited in claim 1, wherein said infusion pump control terminal further comprises an infusion flow rate sensor and a liquid medicine level sensor, wherein said infusion flow rate sensor and said liquid medicine level sensor are connected with said signal input end of said field programmable gate array FPGA respectively.

7. A method for monitoring an infusion pump capable of intelligently easing pain, comprising the following steps of:
(a) eternally connecting an infusion pump control terminal with a human body vital sign sensor and collecting various human body vital sign data of a sufferer by said human body vital sign sensor;
(b) converting a signal by a sensor interface circuit read by said sensor into a signal level which can be recognized by said field programmable gate array FPGA, sending said various human body vital sign data of said sufferer collected to said field programmable gate array FPGA, forwarding said data by said field programmable gate array FPGA to an ARM processor, sending by said ARM processor to a wireless AP through a WIFI communication module, and then transmitting said data to a monitoring server through a local area network; and
(c) drawing up, by a doctor, a best infusion scheme through analysis on said various human body vital sign data of said sufferer at a monitoring server end, and inputting said infusion scheme into said monitoring server through an infusion scheme input module, sending said infusion scheme to said ARM processor of said infusion pump control terminal through said local area network, said wireless AP and said WIFI communication module, generating a control signal by an infusion scheme processing module in said ARM processor on an infusion control device according to said infusion scheme sent by said doctor, and forwarding said control signal to said infusion control device through said field programmable gate array FPGA, so as to realize automatic infusion control of the infusion pump.

8. The method, as recited in claim 7, further comprising a step of inputting an infusion scheme by the sufferer to perform automatic infusion control on said infusion pump.

9. The method, as recited in claim 7, further comprising a step of online perception and feedback, wherein said sufferer inputs online perception and feedback information through an online perception feedback information input module and uploads said online perception and feedback information to said monitoring server.

10. The method, as recited in claim 7, further comprising a step of infusion abnormality warning and a step of infusion completion warning, wherein:
said step of infusion abnormality warning includes an infusion flow rate sensor collecting an infusion flow rate of said infusion pump in real time and judging infusion abnormality when a flow rate value is abnormal, and said field programmable gate array FPGA sending a warning signal to a warning circuit; and
said step of infusion completion warning includes a liquid medicine level sensor collecting the level of residual liquid medicine in said infusion pump in real time and judging infusion completion when said liquid value is lower than a preset threshold, and said field programmable gate array FPGA sending a warning signal to said warning circuit.
